(19) United States
(12) Patent Application Publication
(10) Pub. No.: US 2006/0189599 A1
(43) Pub. Date: Aug. 24, 2006

(54) Treating alcohol and or substance abuse by antagonizing alpha 2 adrenergic receptors with weak dopamine blocking

(76) Inventors: Alan I. Green, Hauover, NH (US); Wing Ming Keung, Wayland, MA (US); Joseph Schildkraut, Chestnut Hill, MA (US); David Chau, White River Junction, VT (US)

Correspondence Address: FISH & RICHARDSON PC, P.O. BOX 1022, MINNEAPOLIS, MN, 55440-1022, US

(21) Appl. No.: 10/531,523

(22) PCT Filed: Oct. 17, 2003
(86) PCT No.: PCT/US03/32852

§ 371 Date: Mar. 15, 2006

Related U.S. Application Data
(60) Provisional application No. 60/419,429, filed on Oct. 18, 2002.

(51) Int. Cl. A61K 31/551; A61K 31/5415; A61K 31/519

(52) U.S. Cl. 514/220; 514/225.8; 514/259.41

(57) ABSTRACT

Certain atypical antipsychotic medications (particularly clozapine) or combinations of medications are useful to treat alcohol or other substance abuse, particularly in the general (non-schizophrenic) population. Generally stated, one aspect of the invention features a method of treating a patient suffering from alcohol or other substance abuse by administering to the patient medication effective to rectify an abuse-associated dysfunction in the DA-mediated brain reward circuit. A second aspect of the invention features administering medication that strongly antagonizes α2 andrenergic receptors and weakly antagonizes dopamine D2 receptors. Preferably, the ratio of α2 receptor blockade to D2o receptor blockade is similar to that of clozapine. The medication may be a single compound (such as clozapine or risperidone), or it may include two or more compounds which together achieve the specified function. For example, the medication may include a first component which weakly blocks the D2 receptor (such as clozapine, quetiapine or ziprasidone or a low dose of another anti-psychotic that is a more potent D2 blocker) and a5 second component (such as clozapine, risperidone or idazoxan) which strongly blocks α2 receptors, particularly the α2C receptor. Cocktails of the two components are also disclosed.

### TECHNICAL FIELD

This invention is in the general field of compositions and treatments for substance abuse, more particularly alcohol abuse.

### BACKGROUND

Alcohol abuse, typically characterized as a maladaptive pattern of alcohol use, leading to clinically significant impairment or distress, is a serious medical and social problem. It has been suggested that agents producing a selective decrease in alcohol drinking in animals, without producing a parallel decrease in water or food intake, are likely to be clinically effective in the treatment of human alcoholism (Myers 1994). Daidzin, the active ingredient of the Chinese herb Radix pureariea (RP), used as a traditional treatment for “alcohol addiction” in China, fits this profile: it decreases alcohol drinking in the golden hamster, without producing a decrease in water or food intake (Keung and Vallee 1993). In contrast, many drugs, including specific serotonergic agonists (e.g., sertraline) and opiate antagonists (e.g., naloxone and naltrexone), that have been shown to inhibit alcohol consumption in animals have also impaired water or food consumption at the same time (Myers 1994).

### SUMMARY

We have discovered that certain atypical antipsychotic medications particularly clozapine) or combinations of medications are useful to treat alcohol or other substance abuse, particularly in the general (non-schizophrenic) population. Generally stated, one aspect of the invention features a method of treating a patient suffering from alcohol or other substance abuse by administering to the patient medication effective to rectify an abuse-associated dysfunction in the DA-mediated brain reward circuit. A second aspect of the invention features administering medication that strongly antagonizes α2 andrenergic receptors and weakly antagonizes dopamine D2 receptors. Preferably, the ratio of α2 receptor blockade to D2 receptor blockade is similar to that of clozapine. Without wishing to bind ourselves to a specific molecular mechanism, it appears that the α2 receptor blockade should at least be directed to the α2C receptor. The medication may be a single compound (such as clozapine or risperidone), or it may include two or more compounds which together achieve the specified function. For example, the medication may include a first component which weakly blocks the D2 receptor (such as clozapine, quetiapine or ziprasidone or a low dose of another anti-psychotic that is a more potent D2 blocker) and a second component (such as clozapine, risperidone or idazoxan) which strongly blocks α2 receptors, particularly the α2C receptor. Clozapine (CLOZ), through its varied actions on serotonergic and noradrenergic neurons (especially its antagonistic effects on α2 andrenergic receptors), coupled with its weak dopamine D2 receptor blocking ability, tends to have a “normalizing” effect on the signal detection capacity of these dysfunctional dopaminergic systems and is therefore useful in the invention.

Other aspects of the invention feature a cocktail that includes the two components.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 compares daily alcohol water, food and total caloric intake in Syrian golden hamsters during 4 baseline days and during 9 days of daily sc injections with either clozapine (CLOZ) or haloperidol (HAL).

FIG. 2 compares daily alcohol consumed during 4 baseline days, the last 4 days of the treatment phase (with clozapine [CLOZ] or haloperidol [HAL]), and during the post-hoc follow-up phase. During the post-hoc phase, animals treated with CLOZ (4 mg/kg) in the treatment phase were given a lower dose (0.2 mg/kg) of CLOZ for the first two days and then vehicle (VEH) for the subsequent days. During this period, alcohol consumption gradually returned toward baseline levels. FIG. 2 indicates alcohol consumption in CLOZ-treated animals for days 21-24 and 30-33 within the post-hoc period. Animals treated with HAL (0.4 mg/kg) in the treatment phase were given HAL at escalating dose (0.6 mg/kg for 2 days, 0.8 mg/kg for 2 days, and 1 mg/kg for 11 days. Alcohol consumption in these animals did not change during the post-hoc period; FIG. 2 indicates alcohol consumption in HAL-treated animals for days 25-28 within the post-hoc period.

### DETAILED DESCRIPTION

As noted the invention generally features methods of treating substance abuse and alcohol abuse in particular. The medications used in the invention are described above. The patients to be treated according to the invention are those with a history or a risk of alcohol abuse, according to DSM-IV.

The compounds to be administered can be formulated into a suitable pharmaceutical preparation by known techniques, for example well known tablet and capsule formulations. Such formulations typically comprise the active agent (or the agent in a salt form) and a pharmaceutically acceptable carrier. As used herein the language “pharmaceutically acceptable carrier” is intended to include any and all solvents, dispersion media, coatings, antibacterial and antifingal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include oral, intravenous, intradermal, subcutaneous, transdermal (topical), transmucosal (e.g. intranasal), and rectal.

By far the most convenient route of administration is oral (ingestion). Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The following experiments specifically demonstrates one embodiment of the invention. The experiment is first summarized and then documented in greater detail

I. Summary of Example

Methods. Twenty adult male Syrian golden hamsters were given access to alcohol in a free choice condition for 24 days prior to drug treatment. Animals were treated with either clozapine (2 mg/kg for 2 days and 4 mg/kg for 7 days) haloperidol (0.2 mg/kg for 2 days and 0.4 mg/kg for 7 days) or vehicle (s.c.) on a daily basis for 9 days and daily consumption of alcohol, water and food was recorded, as was body weight, by a technician blinded to treatment group. Following a 9-day treatment protocol, the animals were followed in a “post-hoc” continued free choice paradigm. The design of the post-hoc period was influenced by the results of the acute treatment protocol. Clozapine-treated animals were followed using vehicle alone to assess the rate at which alcohol drinking returned to baseline. Haloperidol treated animals were followed using increasing doses of haloperidol to assess the effect of these higher doses of haloperidol on alcohol drinking.

Results. Clozapine, but not haloperidol or vehicle, dramatically decreased alcohol consumption to 10% of baseline in Syrian golden hamsters. This effect was accompanied by a modest increase in both water and food intake. During the post-hoc period, alcohol drinking gradually returned toward baseline in the clozapine-treated animals when vehicle was substituted for clozapine. However, animals treated with increasing doses of haloperidol demonstrated no decrease in drinking during this period.

Conclusions. This study demonstrates that the atypical antipsychotic clozapine, but not the typical antipsychotic haloperidol, selectively and reversibly decreases alcohol consumption in the Syrian golden hamster. The effects of antipsychotic drugs (clozapine or other drugs) on alcohol drinking can be assessed in the Syrian golden hamster model or with other animal models, particularly other strains of alcohol drinking rodents, such as the alcohol preferring (P) rat.

II. Detail of Example

This example elucidates the effects of typical and atypical antipsychotics on alcohol consumption. The example is guided by the knowledge of the existence of selected strains of rodents (i.e., alcohol-preferring) that consume substantial amounts of alcohol (McBride and Li 1998). A number of these strains have been used as “animal models” for studying the action of various drugs on alcohol drinking behavior (Myers 1994). One such alcohol-preferring strain is the Syrian golden hamster (Arvola and Forsander 1961; Arvola and Forsander 1963; Kulkosky and Cornell 1979; McCoy et al 1981; Piercy and Myers 1995). This natural, out-bred animal will drink alcohol on a regular basis (under free choice conditions) to maintain a rather predictable blood alcohol level (Keung et al 2000). Under a continuous access regimen, this animal displays a preference for and consumes remarkably large quantities (up to 17 g/kg/day) of ethanol (DiBattista 1986; Keung and Vallee 1993; Kulkosky and Cornell 1979; Piercy and Myers 1995). In experimental settings, the golden hamster will change the volume of alcohol consumed if the alcohol concentration is modified Kulkosky and Cornell 1979); as a result, the ethanol level will remain relatively stable even with the change in the concentration of alcohol. The animal's relatively stable alcohol intake provides a good baseline for study of the effects of medications that might limit alcohol use.

To our knowledge, there have been no studies of the effects of antipsychotic medications on alcohol drinking in the Syrian golden hamster. The Example illustrates the comparative ability of the atypical antipsychotic clozapine, the typical antipsychotic haloperidol and a placebo control vehicle to decrease alcohol drinking in these animals.

Method: Twenty adult male Syrian golden hamsters (weight approximately 90-120 g) were supplied by Harlan Sprague Dawley Inc. (Indianapolis, Ind.). Male hamsters were used because they show stronger preference for ethanol than female hamsters (Arvola and Forsander 1963). Upon arrival, the animals were housed and acclimatized in groups of five in a room maintained at 23° C. on a 12 hr./12 hr. light/dark cycle with ad libitum access to Purina Rodent Laboratory Chow (5001) and spring water (Belmont Spring Water Co., MA). After a week, the animals were transferred to and housed in individual stainless steel metabolic cages (26×18×17.5 cm) equipped with two 50 ml drinking-bottles, one containing Belmont spring water and the other a 15% (v/v) ethanol solution (AAA Alcohol and Chemical Co., KY). The drinking-bottles were placed in bottle-holders equipped with tilted platforms that collect spillage in tubes placed outside of the cages. The positions of the two drinking-bottles on each cage were alternated daily to prevent development of positional preference. The drinks and Purina Chow were provided continuously throughout the course of the experiment. Ethanol, water, food intake, and body weights were measured at 5 pm daily for 24 consecutive days by a research technician. Only animals that drank significant (>8 g/kg/day) and consistent (daily variance<10%) amounts of ethanol in the last 4 days of this period were selected for drug testing. The study was approved by the Harvard Medical Area Standing Committee on Animal Safety.

Medications: Stock clozapine (CLOZ, Novartis Pharmaceuticals) (10 mg/ml) and haloperidol (HAL, Novartis Pharmaceuticals) (1 mg/ml) solutions were prepared by first dissolving the drugs in 0.5 N acetic acid and then adjusting the pH of the solutions to 5.7 using 5 N NaOH. Concentrated drug solutions (Stock solutions) were prepared every other week and stored at −20° C. The vehicle solution was 0.5 M sodium acetate, pH 5.7. Diluted doses for use in hamsters were prepared daily by diluting stock with vehicle.

Experimental Protocol:

Baseline Assessment Period: The last 4 days of the initial free choice period were used to establish baseline values for the subsequent treatment protocol. Based on data from these 4 days, animals were divided into three groups of N=7, N=7 and N=6 animals according to averaged daily alcohol intake, as well as averaged body weight to ensure that animals in every group had similar distributions for these key measures. (Average alcohol consumption (in ml/day) for the three groups were: 15.0±1.1; 15.2±1.1; and 15.5±1.7. Average body weights (in grams) were: 135±5; 134±6; 137±4). Free choice alcohol solution and water were provided continuously throughout the course of the study.

Treatment Assessment Period: The three groups of animals were given either CLOZ (N=7), HAL (N=7) or vehicle (VEH, N=6) by subcutaneous (s.c.) injection (2 ml/kg) on a daily basis at 2 pm. The treatment protocol called for hamsters to be initially given either 2 mg/kg of CLOZ, 0.2 mg/kg of HAL or VEH daily for the first two days. The initial doses of CLOZ and HAL were chosen to equal 20% of those typically used in experiments where the effects of these medications in the CNS of small animals, such as rats, were studied (e.g., Kuroki et al 1999). The plan was to keep this initial dose the same for two days, and then to increase by 2 mg/kg for CLOZ and 0.2 mg/kg for HAL every two days to reach a maximum dose of 10 mg/kg of CLOZ and 1 mg/kg of HAL. Further, the protocol called for doses to be held at a given level for a fall 7 days if any dose (of either medication) caused the alcohol drinking to decrease by more than 75% from baseline. Lastly, the protocol called for the final 4 days of treatment to be used as an endpoint variable for data analysis.

Following the study design, the hamsters were given 2 mg/kg of CLOZ or 0.2 mg/kg of HAL or VEH for 2 days and then received 4 mg/kg of CLOZ or 0.4 mg/kg of HAL for the next 7 days (since the CLOZ treated hamsters had a >75% decrease in alcohol drinking at the 4 mg/kg dose). Assessments of alcohol, water and food consumption, as well as body weight, were made on a daily basis, as indicated above, by a research technician who was blind to group assignment.

Post Hoc Investigations: Post hoc investigations were carried out on these animals to determine: (a) whether the effect of CLOZ on alcohol drinking persists following the cessation of CLOZ treatment; and (b) whether an increased dose of HAL decreases alcohol drinking in these hamsters. Thus, after 9 days of treatment, the dose of CLOZ given to animals treated with CLOZ in the treatment protocol was decreased to 2 mg/kg for 2 days and then to 0 mg/kg (vehicle only) for the next 22 days. During this same time period, the dose of HAL given to HAL treatment animals was increased to 0.6 mg/kg (for 2 days), to 0.8 mg/kg (for 2 days) and to 1 mg/kg (for 11 days). (The 1 mg/kg dose of HAL is within the dose range routinely used in laboratory rodents (Kuroli et al 1999)). Vehicle treated animals continued to receive vehicle during this period.

Data analysis: Separate repeated measures ANOVAs with one between-subject factor (group) and one within-subject factor (time period) were performed for alcohol, water and food intake, and for body weight. Post-hoc pairwise comparisons among the three groups were made using Bonferroni correction. Also, post-hoc pairwise comparisons were made for each of the baseline and treatment days using Tukey HSD tests.

Results:

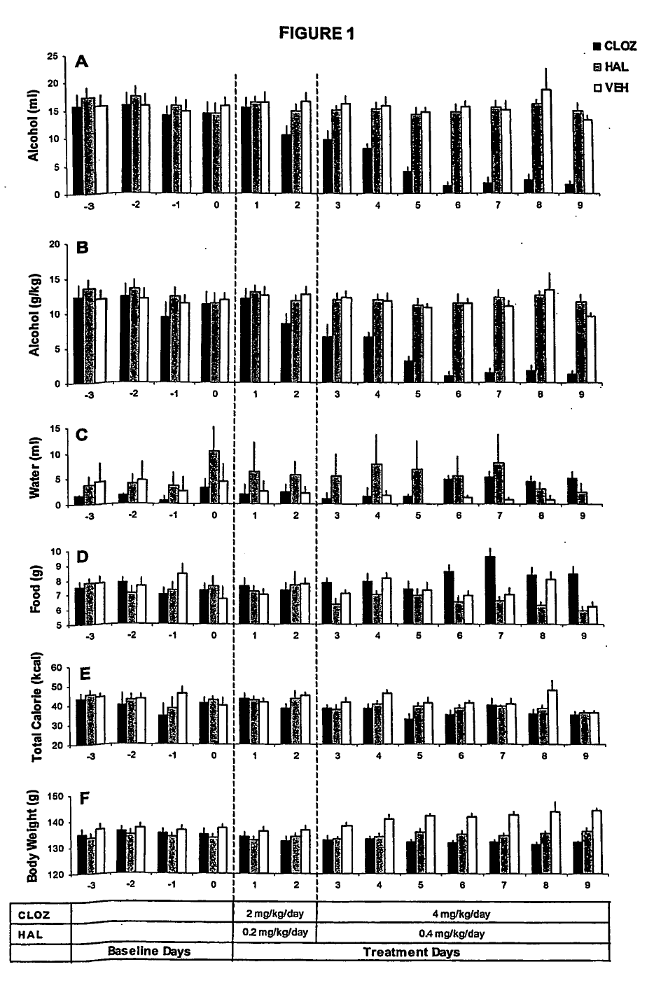

Treatment protocol: FIGS. 1A and 1B demonstrate the course of alcohol intake (in ml/day and g/kg/day) in the CLOZ, HAL and VEH-treated animals. Alcohol drinking began to decrease by treatment day #2, or within 27 hours of the first CLOZ injection (2 mg/kg), and had fallen to 10% of baseline levels by day #6, i.e., after four days of 4 mg/kg/day CLOZ administration. During the same time period, the alcohol drinking was unchanged in the HAL group. The decrease in alcohol consumption in the CLOZ group was significantly different from what was seen in the other two groups (for ml/day: CLOZ vs. HAL, p<0.001 and CLOZ vs. VEH, p<0.001; for g/kg/day: CLOZ vs. HAL, p<0.001 and CLOZ vs. VEH, p<0.001, Bonferroni corrected). Pairwise comparisons of alcohol drinking revealed significant day-by-day differences between CLOZ and HAL, and CLOZ and VEH beginning on treatment day # 3 and continuing for the full 9-day treatment period (FIGS. 1a and 1b)—p<0.002 for all comparisons of daily alcohol drinking for the ml/day variable using Tukey HSD adjustment, and p<0.01 for all comparisons of daily alcohol drinking for the g/kg/day variable. Water drinking increased (from baseline) in the CLOZ animals (p<0.01) (FIG. 1c), but there were no between group differences in water drinking. In addition, compared to baseline, food intake increased in the CLOZ group (p=0.015), while it decreased in the HAL group (p=0.01) and in the VEH group (p=0.06) (FIG. 1d). Despite the increased intake of food and water in the CLOZ group, on average, compared to baseline, the CLOZ group lost weight (p=0.027), while the weight on the HAL group stayed the same and the weight of the VEH group increased (p=0.005). Interestingly, all groups consumed a similar number of total calories per day during the treatment period.

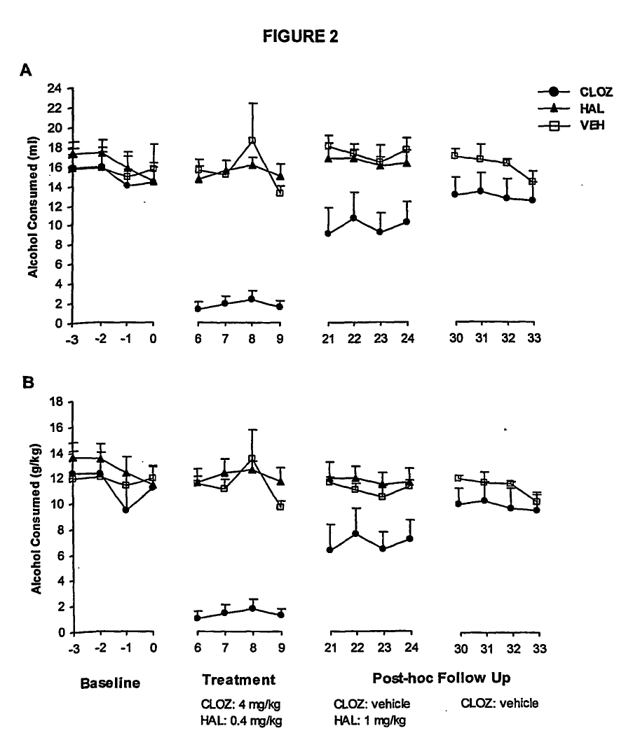

Post-hoc investigations: FIG. 2 shows alcohol consumption during the post-hoc period, compared to baseline and treatment days. Alcohol consumption gradually increased in the CLOZ-treated hamsters over the 24 day post-hoc period (of 2 days of 2 mg/kg and 22 days of vehicle-only treatment), such that by the end of this period (i.e., the last 4 days of the period), drinking essentially returned to levels comparable to those during the baseline period (for ml/day, p=0.1; for g/kg/day, p=0.07). Water and food consumption returned to pre-CLOZ levels during this time. Regarding the HAL group, there was no change in alcohol drinking (as assessed by mg/kg or ml consumed per day) over the 15-day post hoc period (with 2 days of 0.6 mg/kg, 2 days of 0.8 mg/kg and 11 days of 1 mg/kg daily haloperidol injections). Throughout the post hoc investigation study, all three groups of animals consumed a similar number of calories per day.

Discussion:

This Example demonstrates that the administration of the atypical antipsychotic CLOZ, but not the typical antipsychotic HAL, decreases alcohol consumption by the Syrian golden hamster. CLOZ (4 mg/kg) reduced alcohol intake by more than 90% during treatment. During the 24 day post hoc assessment period, with 2 days of low dose CLOZ and 22 days of VEH treatment only, the effect on alcohol drinking gradually reversed, such that by the end of the period it was not significantly different from the baseline level of drinking. By contrast to the effect seen with CLOZ, over a full 24 days of treatment with HAL (with dose ranging from 0.2 mg/kg to 1 mg/kg), and including 11 days at the highest dose of HAL during the post-hoc period, there was no evidence of a decrease in alcohol use.

Importantly, while alcohol drinking decreased with CLOZ, food and water intake increased, and total caloric intake was constant. This would appear to indicate that the effect on alcohol consumption by CLOZ is specific, and not a generalized effect on drinking or eating.

The Syrian Golden Hamster may be a particularly good animal to use for assessing the ability of antipsychotic drugs to reduce alcohol drinking. First, like patients with schizophrenia, the animal consumes alcohol on a regular basis (Arvola and Forsander 1961; Arvola and Forsander 1963; Kulkosky and Cornell 1979; McCoy et al 1981; Piercy and Myers 1995). Second, also as with the patients (who tend to drink moderately on a regular basis and develop comorbid alcohol abuse much more frequently than they develop alcohol dependence (Drake et al 1989; Lehman et al 1996; Test et al 1989)), despite regular drinking by the hamsters, physiological withdrawal from the alcohol (as assessed by the sound-induced seizure technique) has not been observed (McMillan et al 1977). And third, as with the patients, it is clear that alcohol produces central nervous system effects in the hamster—changes in central serotonin metabolism (Keung, 2000) and circadian rhythm (Mistlberger, 92), reduced leucine-enkephalin expression in the basal ganglia (Blum et al 1982), and impairment in aversive learning (Harris, 1979) have all been described during free choice alcohol drinking in these animals.

Other atypical antipsychotics—risperidone, olanzapine, quetiapine and ziprasidone—may be assessed for use in the invention.

Without wishing to bind ourselves to any specific mechanism of action, we conclude that, even in non-schizophrenic patients, deficiencies in the DA-mediated mesocorticolimbic circuits underlie alcohol use and that CLOZ but not HAL will decrease alcohol use because it ameliorates these circuit deficiencies. Since some alcohol-preferring animals (especially the alcohol preferring “P rat”) have been noted to have deficiencies in DA mediated mesolimbic circuits (McBride and Li 1998), the ability of CLOZ but not HAL to limit alcohol drinking in the Syrian golden hamster suggests the possibility that CLOZ limits alcohol use through amelioration of a mesolimbic dysfunction. DA circuits of the golden hampster can be directly studied, and the comparative effects of CLOZ and HAL on alcohol drinking in the P rat can be investigated to further elucidate the neurobiologic basis of the effects of CLOZ on alcohol consumption. We propose that, in patients with alcohol or substance abuse, (a) there is a dysfunction in their dopamine (DA) mediated mesocorticolimbic reward pathways (with impaired signal detection capacity); (b) this “reward dysfunction” underlies alcohol/substance use in this population; and (c) the primary biological effects of alcohol and other substances may involve a transient amelioration of the dysfunction in this brain reward system. Clozapine, through its various actions on multiple neurotransmitter systems, particularly its potent blockade of α2 noradrenergic receptors, its striking increase in norepinephrine levels, as well as its weak blockade of dopamine D2 receptors, may tend to have a normalizing effect on the signal detection capacity of this dysfunctional mesocorticolimbic brain reward circuit.

The findings from our hamster study support the use of clozapine (or another medication with a similar spectrum of effects on α2, and most partiularly α2C, and D2 receptors) to treat alcohol abuse based on the concept that individuals with alcohol abuse or dependence have a dysfunction in their DA-mediated brain reward circuit that on some levels resembles the dysfunction in patients with schizophrenia, and that a medication that could correct this dysfunction might be an effective treatment for alcohol abuse or dependence.

It is important to recognize that such a treatment might also be an important treatment for substance abuse in general, since most substances of abuse act on DA circuits in a manner quite similar to that of alcohol. Other such substances of abuse are: cannibus, amphetamines and cocaine.

### REFERENCES

Albanese M, Khantzian E, Murphy S, Green A (1994): Decreased substance use in chronically psychotic patients treated with clozapine. Am J Psychiatry 151:780-1.
Alterman A, Erdlen F, Murphy E (1981): Alcohol abuse in the psychiatric hospital population. Addict Behav 6:69-73.
Arvola A, Forsander O (1961): Comparison between water and alcohol consumption in six animal species in free choice experiments. Nature 191:819-820.
Arvola A, Forsander O (1963): Hamsters in experiments of free choice between alcohol and water. Q. J. Stud. Alcohol 24:591-597.
Blum K, Briggs A, Elston S, DeLallo L, Sheridan P (1982): Reduced leucine-enkephalin-like immunoreactive substance in hamster basal ganglia after long-term ethanol exposure. Science 216:1425-1427.
Buckley P, McCarthy M, Chapman P, Richman C, Yamamoto B (1999): Clozapine treatment of comorbid substance abuse in patients with schizophrenia. Schizophr Res 36:272.
DiBattista D (1986): Voluntary ethanol consumption and obesity in golden hamsters. Physiol Behav 36:41-45.
Drake R, Osher F, Wallach M (1989): Alcohol use and abuse in schizophrenia. A prospective community study. J Nerv Ment Dis 177:408-414.
Drake R, Xie H, McHugo G, Green A (2000): The effects of clozapine on alcohol and drug use disorders among patients with schizophrenia. Schizophr Bull 26:441-9.
Freed E (1975): Alcoholism and schizophrenia: the search for perspectives. A review. J Stud Alcohol 36:853-81.
Green A, Zimmet S, Strous R, Schildkraut J (1999): Clozapine for comorbid substance use disorder and schizophrenia: do patients with schizophrenia have a reward-deficiency syndrome that can be ameliorated by clozapine? Harv Rev Psychiatry 6:287-96.
Green A, Burgess E, Dawson R, Zimmet S, Strous R (in press) Alcohol and cannabis use in schizophrenia: effects of clozapine vs. risperidone. Schizophr Res.
Keung W, Kunze L, Li D, Lazo O (2000): Volitional ethanol consumption affects overall serotonin metabolism in Syrian golden hamsters (Mesocricetus auratus). Biochem Biophys Res Commun 271:823-830.
Keung W, Vallee B (1993): Daidzin and daidzein suppress free-choice ethanol intake by Syrian golden hamsters. Proc Natl Acad Sci USA 90:10008-12.
Kulkosky P, Cornell N (1979): Free-choice ethanol intake and ethanol metabolism in the hamster and rat. Pharmacology Biochemistry & Behavior 11:439-444.
Kuroki T, Meltzer H, Ichikawa J (1999): Effects of antipsychotic drugs on extracellular dopamine levels in rat medial prefrontal cortex and nucleus acciumbens. J Phanracol Exp Ther 288:774-781.
Lehman A, Myers C, Dixon L, Johnson J (1996): Detection of substance use disorders among psychiatric inpatients. J Nerv Ment Dis 184:228-233.
McBride W, Li T (1998): Animal models of alcoholism: neurobiology of high alcohol-drinking behavior in rodents. Crit Rev Neurobiol 12:339-369.
McCoy G, Haisley A, Powchik P, Tambone P (1981): Ethanol consumption by Syrian golden hamsters. Food intake and blood ethanol levels. J Stud Alcohol 42:508-513.
McMillan D, Ellis F, Frye G. Pick J (1977): Failure of signs of physical dependence to develop in hamsters after prolonged consumption of large doses of ethanol. Pharmacology Biochemistry & Behavior 7:55-57.
Myers R (1994): New Drugs for the Treatment of Experimental Alcoholism. Alcohol 11:439-451.
Piercy K, Myers R (1995): Female Syrian golden hamster: drinking of high concentrations of ethanol aversive to other mammals. Alcohol 12:207-211.
Regier D, Farmer M, Rae D, et al (1990): Comorbidity of mental disorders with alcohol and other drug abuse. Results from the Epidemiologic Catchment Area (ECA) Study. JAMA 264:2511-8.
Test M, Wallisch L, Allness D, Ripp K (1989): Substance use in young adults with schizophrenic disorders. Schizophr Bull 15:465-476.
Zimmet S, Strous R, Burgess E, Kohnstamm S, Green A (2000): Effects of clozapine on substance use in patients with schizophrenia and schizoaffective disorder: a retrospective survey. J Clin Psychopharmacol 20:94-8.

A number of embodiments of the invention have been described. Nevertheless, it will be understood that various modifications may be made without departing from the spirit and scope of the invention. Accordingly, other embodiments are within the scope of the following claims.

1. A method of treating a patient suffering from substance abuse comprising administering to the patient medication that includes a dose of clozapine, risperidone or both, effective to reduce abuse.

2. A method of treating a patient suffering from substance abuse comprising administering to the patient medication effective to strongly antagonize α2 andrenergic receptors and to weakly antagonize dopamine D2 receptors.

3. A method of treating, a patient suffering from substance abuse comprising administering to the patient medication effective to rectify an abuse-associated dysfunction in the DA-mediated brain reward circuit.

4. The method of any one of claims 1-3 in which the patient is not schizophrenic.

5. The method of any one of claims 1-3 in which the medication comprises clozapine.

6. The method of claim 3 in which the medication comprises:
a first component which weakly blocks the D2 receptor; and
a second component which strongly blocks α2 receptors.

7. The method of claim 6 in which the first component is selected from clozapine, risperidone, olanzapine, quetiapine and ziprasidone.

8. The method of claim 6 or 7 in which the second component is idazoxan, or another α2 receptor antagonist.

9. The method of claim 2 in which the medication is formulated as a single dose comprising both the first and the second components.

10. The method of claim 2 or 3 in which the medication is characterized by a ratio of α2 blockade: D2 receptor blockade similar to that of clozapine.

11. The method claim 10 in which the medication is characterized by a ratio of α2C blockade: D2 receptor blockade similar to that of clozapine.

12. The method of claim 6 in which the medication strongly blocks the a2C receptor.

13. A cocktail comprising
a first component which weakly blocks the D2 receptor; and
a strong α2 receptor antagonist.

14. The cocktail of claim 13 in which the first component is selected from clozapine, risperidone, olanzapine, quetiapine and ziprasidone.

15. The cocktail of claim 13 or 14 in which the second component is idazoxan, or another α2 receptor antagonist.

16. The cocktail of claim 13 or 14 in which the cocktail is characterized by strong blockade of the α2C receptor.

17. The cocktail of claim 13 or 14 in which the cocktail is characterized by a ratio of α2 blockade: D2 receptor blockade similar to that of clozapine.

18. The method claim 12 in which the cocktail is characterized by a ratio of α2C blockade: D2 receptor blockade similar to that of clozapine.

19. The method of any one of claims 1-3 wherein the substance is alcohol.
